(19) United States
(12) Patent Application Publication
(10) Pub. No.: US 2009/0324506 A1
(43) Pub. Date: Dec. 31, 2009

(54) Sprayable skin protectant formulation and method of use

(76) Inventors: Jeffery Richard Seidling, Neenah, WI (US); Scott W. Wenzel, Neenah, WI (US); Sarah Anne Lemke, Appleton, WI (US); Kroy Donald Johnson, Neenah, WI (US); Stacy Averic Mundschau, Weyauwega, WI (US)

Correspondence Address: KIMBERLY-CLARK WORLDWIDE, INC.;Tara Pohlkotte, 401 NORTH LAKE STREET, NEENAH, WI, 54956, US

(21) Appl. No.: 12/217,042

(22) Filed: Jun. 30, 2008

(51) Int. Cl. A61K 9/12; A61P 17/02

(52) U.S. Cl. 424/45

(57) ABSTRACT

The present disclosure is directed to a skin protectant formulation that is designed to be sprayed onto human skin for the purpose of protecting the skin. In particular, an exemplary formulation of the present disclosure contains at least a carrier, a structurant and a skin protectant agent. An exemplary formulation contains a carrier comprising at least water, a structurant comprising at least a laponite clay, and a skin protectant agent comprising petrolatum. Typically, the skin protectant formulation will comprise at least about 30 wt. % of a skin protectant agent. A continuous spray dispenser may be used to apply the skin protectant formulation to reduce the need for rubbing as the treated area can be covered by a mist of the skin protectant formulation requiring no need to rub for application or small droplets that do not require extensive time for rubbing in.

### FIELD

The present disclosure generally relates to topical skin protectant formulations and methods of treating fragile skin with skin protectant formulations. More particularly, sprayable skin protectant formulations having a structurant and at least about 30% (by weight of the formulation) of a skin protectant agent, wherein the skin protectant formulation may be used without clogging or obstructing the opening of the dispenser are disclosed.

### BACKGROUND

Consumer products are often applied to human skin to protect and/or treat skin. These consumer products can be used to protect and/or treat skin in connection with various ailments, including but not limited to diaper rash; minor burns; cuts; scrapes; sunburn; chaffed, chapped, cracked, or windburned skin or lips; skin irritation; and oozing and/or weeping of skin caused by poison ivy, poison oak, and/or poison sumac.

Skin protectants are heavily used by the elderly population to alleviate some of these skin conditions. For example, elderly skin is often dry, itchy, frail and easily torn or bruised. In addition to this, incontinence and occlusive incontinence garments can cause rashes and other skin problems. Elderly consumers may not be able to squeeze a bottle very effectively and rubbing or otherwise manipulating the skin can cause tearing and bruising.

Fragile skin is difficult for an individual or caregiver to touch since it is easily torn and bruised. Also, compromised skin, or skin that is damaged in some way both need to be cleaned and/or moisturized in a sensitive manner. However, conventional delivery of these skin protectants may be anxiety ridden for a caregiver and cause additional discomfort or irritation for the sufferer.

Skin protectants are also often used on children to alleviate skin problems such as diaper rash and eczema. The skin of infants and children is known to be highly sensitive. Additionally, children are difficult to keep still and often one hand is needed to hold the child and the other hand is needed to apply the formulation to the site of irritation. Cream and ointment type dispensing typically require two hands to dispense the formulation, one hand to squeeze the tube and the other hand to receive the formulation for application onto the child.

One commonly used skin protectant is petrolatum. Barrier products containing oily substances such as petrolatum may feel greasy, may be difficult to apply because of their high viscosity, and may not be easily removed from hands that apply the products or from the infant's skin. Clean-up of these products from the hands and from the infant's skin may be regarded by some as time-consuming, messy, and inconvenient. Additionally, petrolatum is highly viscous and is not easily sprayable. Highly viscous fluids tend to sputter and clog the opening of dispensers when sprayed.

Other skin protectants have been utilized before in a sprayable composition. For example, several commercially available sprayable formulations containing dimethicone have been utilized as a skin protectant. However, use of a high petrolatum skin protectant provides added benefit over dimethicone due to higher add-on and more substantivity to the skin.

Therefore, a need exists for a skin protectant formulation that can be applied as an even mist onto the skin that would ensure complete coverage without rubbing or would require less rub-in time as the formulation is dispensed as small droplets.

### SUMMARY

It has been found that a formulation can be provided having a skin protectant agent for utilization with a continuous spray dispenser without clogging the dispenser. Particularly, in one aspect, the skin protectant formulation includes a combination of a carrier, a structurant, and a skin protection agent.

Additionally, the skin protectant formulation provides an even and efficient means to treat the consumer's skin. In an exemplary aspect, the use of a continuous spray dispenser provides small droplets of the skin protectant formulation reducing the need to rub the formulation into the skin or reducing rub-in time. To allow for spraying the formulation, the structurant provides a network trapping the skin protectant agent within the network allowing the formulation to be easily sprayed. The structurant keeps the skin protectant agent uniformly distributed throughout the formulation, thus minimizing the likelihood of clogging the nozzle. Accordingly, the sprayable skin protection formulation is adapted to be sprayed on fragile or sensitive skin.

In accordance with the present disclosure, a sprayable skin protection formulation containing at least a carrier, a structurant, and at least about 30 wt. % of a skin protectant agent is disclosed. In another aspect, the skin protectant formulation contains between about 30 wt. % to about 60 wt. % of a skin protectant agent.

In one exemplary aspect, the skin protectant agent is petrolatum. In other aspects, the skin protectant agent may be lanolin or cocoa butter.

The structurant may be selected from an acrylate, a clay, a starch, a modified cellulose, a natural gum, a wax, colloidal particles, and/or mixtures thereof. In an exemplary aspect, the clay may be selected from bentonite, laponite, hectorite, montmorillonite, beidelite, saponite, stevensite, magnesium aluminum silicate, other aluminum silicates, as well as various other natural and/or synthetic clays, and combinations thereof. In an exemplary aspect, the structurant is laponite. The structurant could also be a starch; a modified cellulose, the modified cellulose comprising ethyl cellulose, hydroxypropyl cellulose, carboxymethyl cellulose, hydroxypropyl methyl cellulose, hydroxyethyl methyl cellulose, hydroxyethyl cellulose, or mixtures thereof; or a natural gum, the natural gum comprising guar gum, pectin, gum Arabic, locust bean gum, xanthan gum, carrageenan gum, or mixtures thereof.

In general, the skin protectant formulation contains between about 0.01 wt. % to about 10 wt. % of a structurant, more particularly, about 0.05 wt. % to about 5 wt. % of the structurant, and even more particularly about 0.1 wt. % to less than about 5 wt. % of the structurant.

The sprayable skin protection formulation contains a carrier. In one exemplary aspect, the carrier is a hydrophilic carrier such as water. The carrier could also be a hydrophobic carrier or an emulsion such as an oil-in-water emulsion. In this case the formulation will require emulsifiers, hydrophilic carriers and hydrophobic carriers.

In another aspect, the sprayable skin protection formulation may also contain a treatment agent selected from the group consisting of emollients, humectants, natural fats and oils, anti-irritants, antimicrobial agents, antioxidants, anti-parasitic agents, antipuritics, antifungals, antiseptic actives, keratolytic actives, anti-stinging agents, anti-reddening agents, astringents, biological actives, deodorants, external analgesics, film formers, fragrances, skin condition agents, skin exfoliating agents, skin soothing ingredients, sunscreens and combinations thereof.

The skin protectant formulation contains between about 0.01 wt. % to 40 wt. % of the treatment agent, more particularly, between about 0.05 wt. % to about 30 wt. % of the treatment agent.

A method of treating fragile and sensitive skin is also disclosed. To treat fragile skin, a sprayable skin protection formulation as described above is provided. This formulation is then continuously sprayed onto an area of human skin for protecting the skin. The method applies droplets of the skin protection formulation onto the skin for application without the need for excessive rubbing.

Other objects and features will be in part apparent and in part pointed out hereinafter.

### DETAILED DESCRIPTION

It is to be understood by one of ordinary skill in the art that the present discussion is a description of exemplary embodiments only, and is not intended as limiting the broader aspects of the present disclosure.

The present disclosure is directed to a skin protectant formulation that is designed to be sprayed onto human skin for the purpose of protecting the skin. In particular, an exemplary formulation of the present disclosure contains at least a carrier, a structurant and a skin protectant agent. Typically, the skin protectant formulation will comprise at least about 30 wt. % and more preferably from at least about 30 wt. % to about 60 wt. % of the skin protectant agent. Additionally, the structurant can ensure that the skin protectant agents do not agglomerate or otherwise settle out of solution while also providing a thixotropic effect when shear is applied to the formulation. The thixotropic nature of the formulation results in a decrease in the viscosity of the formulation allowing the product to be sprayed. In an exemplary aspect, a continuous spray dispenser is used to apply the skin protectant formulation to reduce the need for rubbing as the treated area can be covered by a mist of the skin protectant formulation requiring no need to rub for application or small droplets do not require extensive time for rubbing in.

The skin protectant formulation of the present disclosure is sprayable. By sprayable it means that the skin protectant formulation is dispensed through a hand-held spray dispenser by pressing a dispensing button to spray the formulation onto the skin. Any conventional spray dispenser may be used to dispense the skin protectant formulation including aerosol or pressurized propellant dispensers, motor driven pump dispensers, or dispensers using manual spray pump mechanisms.

In a preferred embodiment, the skin protectant formulation of the present disclosure may be utilized with a continuous spray dispenser. Continuous spray, or continously sprayable, technology is meant to indicate that the formulation provides any-angle spraying and uniform coverage. An example of a continuous spray dispenser would include a flexible, expandable container adapted to receive the skin protectant formulation. The flexible container is removably surrounded by a rigid exterior housing or canister, which is provided with an air-tight seal. The canister is sealed prior to filling the flexible container with the skin protectant formulation, so that air is trapped within the canister in the volume unoccupied by the flexible container. When the flexible container is filled with the skin protectant formulation, the container expands, thereby compressing the air within the canister. While maintaining complete separation from the skin protectant formulation, this compressed air acts as a propellant. The compressed air then acts against the flexible container to uniformly propel the skin protectant formulation from the container. In this example, there is no need to pump the spray like conventional spray dispensers to distribute the formulation onto skin. This is advantageous in limiting pain for those with limited dexterity or arthritis.

In another exemplary continuous spray dispenser, the container may include a pump that is integral with the cap on the dispenser. In this example, the air is compressed in the canister not when sealing the canister, but by pumping air into the canister to provide compressed air as a propellant. The compressed air added by a consumer then acts against the flexible container to uniformly propel the skin protectant formulation out of the container.

Continuous spray technology is well known in the art. Suitable commercially available continuous spray dispensers for use with the skin protectant formulation can include, for example, the 12HS Dry Spray Dispenser commercially available from Rexam Airspray or the bag-on-valve dispenser commercially available from ColepCCL.

It is not believed that any special design for the hand-held continuous spray dispenser is needed. Specific dimensions and/or materials of construction are easily discernible and defined by one skilled in the art based on the chemical and physical properties of the formulation to allow for appropriate dispensing of the formulation.

Combining continuous sprays and fragile skin-friendly formulations provides a means to deliver controlled dosing of formulations to the skin in a non-contact and even coverage manner. The delivery of the skin protectant formulations of the present disclosure is ideal for fragile skin on any area of the body in a painless, pleasant manner and to prevent further damage by rubbing damaged skin. Continuous spray technology provides easy to dispense packaging in a mist form. As elderly skin is very fragile, rubbing the skin can cause bruising and skin tearing which can lead to infection and discomfort for the elderly person. Due to the structurant holding the skin protectant agent uniformly in the solution and allowing for spraying under high shear, a continuous spray can cover a treated area with a fine mist of small droplets containing the skin protectant formulation coating the skin evenly. Therefore, the skin protectant agent is evenly distributed on the skin minimizing the need for extensive rub-in time. This reduces the risk for bruising and tearing of elderly or sensitive skin. In addition this technology provides the ability to spray an even coverage of formulations at any angle to help reach “hard to reach areas” such as in the perineal region.

This dispensing method is particularly suitable for those with compromised dexterity or strength. An elderly person or caregiver would easily be able to spray the formulation onto the skin. Additionally, a continuous spray system can be directly applied to the affected area of a child using only one hand and requiring minimal to no touching of the tender area of the child following application.

Furthermore, existing sprayable skin protectant formulations are commonly dispensed by spray pumps which cause the application of a large concentration of the formulation in one area. Such a problem often results from the use of conventional skin protectant formulations, especially in the area of initial application from where the skin protectant formulation is spread.

A significant drawback to using spray pumps, however, is that to effectively release liquid from such containers, the user must maintain the container in an upright position or substantially close to a 90-degree angle, or perpendicular to the skin. Additionally, spray pumps require the user to repeatedly press on a nozzle and then release, thereby resulting in a pause between each spray of liquid which increases the opportunity for uneven application of a formulation.

Using the continuous spray dispenser allows the formulation to be uniformly applied to the skin in a light mist form, which quickly dries on the skin without rubbing if that is required. Since the structurant is maintaining the skin protection agent uniformly in the formulation, the skin protectant agent is also uniformly applied to the skin. Alternatively, minimal rubbing may be used to spread or dry the formulation on the skin which is much easier on sensitive skin.

The delivery of formulations in the continuous spray form would be additionally beneficial to a caregiver who was applying the formulation to a sufferer, or to the sufferer themselves who may have difficulty applying lotion to particular body areas in an even manner.

As mentioned above, the skin protectant formulation contains at least one skin protectant agent. Typically, the skin protectant formulation will comprise at least about 30 wt. % more preferably from at least about 30 wt. % to about 60 wt. % of the skin protectant agent. These skin protectant agents could include mineral oil, dimethicone, zinc oxide, allantoin, calamine, kaolin, petrolatum, white petrolatum, cod liver oil, lanolin, talc, topical starch, aluminum hydroxide gel, cocoa butter, glycerine, shark liver oil, zinc acetate, zinc carbonate, and combinations thereof.

A preferred skin protectant agent utilized with the formulation of the present disclosure is petrolatum. A preferred petrolatum would be grades of petrolatum available from Sonneborn Refined Products sold under the trade name Sonnecone®. These grades of petrolatum are USP petrolatum, but instead of the residual greasy feel, provide a silicone feel on the skin. The viscosity of Sonnecone® is about 150,000 centipoise at 25° C. An exemplary viscosity range for the petrolatum used as a skin protectant agent of the present disclosure is between about 20,000 centipoise and 600,000 centipoise at 25° C. A preferred embodiment uses the Sonnecone® CM petrolatum as a skin protectant in the formulation to provide protective benefits to the skin.

Petrolatum acts as a very good skin protectant, but is not known to have previously been utilized in continuous spray formulations as the viscosity of the petrolatum is quite high. Additionally, the percentage of petrolatum necessary to achieve a skin protective benefit is too high. Thus, use of petrolatum may clog the spray pumps. The result would be a spray that either does not spray at all or sprays inefficiently by sputtering through the orifice and leaving very uneven coverage on the skin. If any formulations did include petrolatum, the formulations did not include petrolatum in sufficient amounts to provide the necessary skin protectant benefits. It has been unexpectedly discovered that a formulation having a structurant with the skin protectant agent of the present disclosure is capable of being sprayed easily and efficiently and delivers extremely fine droplets of the skin protectant agent and even coverage on the skin.

To provide a sprayable formulation, the structurant may act as a thixotropic agent, gradually reducing of viscosity with constant shear (finite amount of time required to change from high viscosity to low viscosity at a constant shear rate). The thixotropic agent of the present disclosure provides a reduction in viscosity in the skin protectant formulation with increasing shear rate to provide an immediate change proportional to the amount of shear allowing for a sprayable formation, while also allowing the formulation to remain sufficiently viscous to suspend the skin protectant particles therein when under low or no shear. Therefore, the composition is formulated such that the structurant creates a network that traps the skin protectant within the network and keeps the skin protectant from coalescing when no shear is applied to the formulation. In other words, in order to maintain the skin protectant agents dispersed throughout the formulation, the skin protectant agent may be added to the formulation in the presence of a structurant. The structurant can ensure that the skin protectant agents do not agglomerate or otherwise settle out of solution. Additionally, when shear is applied to the formulation, the thixotropic nature of the structurant thins to a milk-like consistency and is able to be sprayed. Furthermore, when the formulation hits the skin and there is no applied shear, the formulation will again thicken such that it will not run off the surface of the skin and therefore create an even coating on the skin.

Previous attempts to create sprayable petrolatum formulations have involved the use of encapsulated vesicles to entrap the petrolatum skin protectant. These are complicated wall forming structures that involve a lipid phase and weighting agents. By simply adding a structurant to the liquid formulation of the present disclosure, a matrix or network is created that provides less viscosity to the formulation under shear. The skin protectant formulation of the present disclosure provides a simple solution to allow for a sprayable petrolatum formulation.

In one embodiment, for instance, the structurant may comprise an acrylic polymer, such as an acrylate, that is designed to suspend the dyes and to stabilize and/or thicken the skin protectant formulation. For instance, in one embodiment, the structurant may comprise CARBOPOL 980 polymer available from Noveon, Inc. CARBOPOL 980 polymer is a homopolymer of acrylic acid crosslinked with an allyl ether of pentaerythritol, an allyl ether of sucrose, or an allyl ether of propylene.

In other aspects, non-acrylic based suspending agents may be used. For instance, the suspending agent may comprise a clay, a starch, a cellulose, a gum, a wax, a fatty acid, a fatty alcohol, colloidal particles, or other non-acrylic based water soluble polymeric thickeners. The structurant can be added in an amount sufficient to suspend the dyes and to otherwise stabilize the composition.

For instance, in one embodiment, clay particles may be added to the skin protectant formulation as the structurant. The clay particles may comprise, for instance, any suitable phyllosilicate material. The clay particles, for instance, can generally have a particle size of less than about 2 microns. The structurant of the present disclosure may comprise water-swellable clay. A variety of clays are suitable for use as structurants in the skin protectant formulation described herein including, for example, bentonite, laponite, hectorite, montmorillonite, beidelite, saponite, stevensite, magnesium aluminum silicate, other aluminum silicates, as well as various other natural and/or synthetic clays, and combinations thereof.

In another embodiment, the structurant may comprise a starch, which includes starch derivatives. Starches are generally available from plants, such as corn, rice or tapioca and comprise a complex carbohydrate. Starch derivatives generally include starches that have been hydrolyzed into simpler carbohydrates by acids, enzymes, or a combination of the two.

Another example of a structurant that may be used in the present disclosure includes cellulose materials, particularly modified cellulose. Modified cellulose is generally referred to as cellulose where the hydroxyl groups of the cellulose are partially or fully reacted with various chemicals. Modified celluloses include cellulose esters and cellulose ethers. Cellulose structurants particularly well suited for use in the present disclosure include ethyl cellulose, hydroxypropyl cellulose, carboxymethyl cellulose, hydroxypropyl methyl cellulose, hydroxyethyl methyl cellulose, hydroxyethyl cellulose, and combinations thereof.

In still another embodiment, the structurant may comprise a natural gum. Natural gums well suited for use in the present disclosure include guar gum, carrageenan, gum Arabic, locust bean gum, xanthan gum, and mixtures thereof. Natural gums also include any derivatives of the above gums. For instance, hydroxypropyl guar gum may also be used.

In a preferred embodiment, the structurant can include, but not be limited to, laponite clay, such as Laponite XLG commercially available from Southern Clay Products. Laponite XLG is a synthetic, layered clay, similar to natural smectites.

The laponite synthetic layered clays (sodium magnesium silicate) described above are extremely thixotropic. For example, a 2% water gel made with synthetic layered clay shows a change in viscosity from nearly 10⁸ cP to less than 30 cP under a shear rate of 10³ s⁻¹. As a result, use of the disclosed laponite synthetic layered clays as a structurant allows the formulation of the present disclosure to be sprayed. In the present disclosure, the skin protectant agent is dispersed as small droplets in the laponite synthetic layered clay system.

Typically, the skin protectant formulation will comprise from about 0.01 wt. % to about 10 wt. % and more preferably from about 0.05 wt. % to about 5 wt. % of a structurant. In one embodiment, the skin protectant formulation may comprise from about 0.1 wt. % to less than about 5 wt. % of the structurant.

As noted above, the skin protectant formulation of the present disclosure may comprise a hydrophilic carrier. Typically, any hydrophilic carrier known by one skilled in the art can be used. Preferably, water is utilized as the carrier in the skin protectant formulation.

Other suitable hydrophilic carriers include glycerin, glycerin derivatives, glycols, such as polyethylene glycols and derivatives thereof, polypropylene glycols, propylene glycol, butylene glycol, ethoxydiglycol, and the like, and combinations thereof. Further examples of suitable carriers include those described in CTFA, International Cosmetic Ingredient Dictionary and Handbook, 12th Ed. (2008), which is hereby incorporated by reference to the extent that it is consistent herewith. In this case, the petrolatum is dispersed within a thickened system.

The carrier could also be an emulsion such as an oil-in-water emulsion, including emulsifiers, hydrophilic and hydrophobic carriers. Suitable hydrophilic carriers include, but are not limited to, propylene glycol, butylene glycol, dipropylene glycol, glycerin, glycereth-18 ethylhexanoate, glycereth-18, betaine, diglycerin, glycol, inositol, meadowfoamamidopropyl betaine, ethyl alcohol, isopropyl alcohol, polyethylene glycol with varied molecular weights, sorbitol, xylitol, urea, tripropylene glycol, sodium PCA, glycereth-7 glycolate, diglycereth-7 malate, 2,3-butanediol, propanediol, xylose, almond oil PEG-6 esters, apricot kernel oil PEG-6 esters, argan oil PEG-8 esters, argan oil polyglyceryl-6 esters.

Suitable hydrophobic substances for use as a carrier include, but are not limited to, PEG-3 dimethicone, PEG/PPG-20/23 dimethicone, PEG-8 dimethicone, cyclomethicone, dimethcione, cetyl dimethicone, caprylyl methicone, ethyl trisiloxane, trimethylsiloxyamodimethicone, stearyl dimethicone, silicones with polypropylene glycol functionality such as PPG-12 dimethicone, silicones with polyethylene glycol functionality such as PEG-8 trisiloxane, PEG-10 dimethicone and silicones which combine both functionalities in varying ratios such as PEG/PPG-5/3 trisiloxane, PEG/PPG-8/26 dimethicone, PEG/PPG-20/15 dimethicone, bis-PEG-4 dimethicone, bis-PEG-12 dimethicone, bis-PEG/PPG-14/14 dimethicone, bis-PEG/PPG-18/6 dimethicone, bis-PEG/PPG-20/20 dimethicone, butylene glycol behenate, butylene glycol diisononanoate, butylene glycol laurate, butylene glycol myristate, butylene glycol oleate, butylene glycol palmitate, butylene glycol stearate, butyl isostearate, butyl myristate, butyloctyl behenate, butyloctyl benzoate, butyloctyl cetearate, butyloctyl palmitate, butyl oleate, butyl stearate C₁₄₋₁₅ alcohols, C₁₈₋₂₈ alkyl acetate, C₁₂₋₁₅ alkyl benzoate, C₁₆₋₁₇ alkyl benzoate, C₃₀₋₄₅ alkyl cetearyl dimethicone crosspolymer, C₃₂ alkyl dimethicone, C₃₀₋₄₅ alkyl dimethicone/polycyclohexene oxide crosspolymer, C₁₂₋₁₃ alkyl ethylhexanoate, C₁₂₋₁₅ alkyl ethylhexanoate, C₁₄₋₁₈ alkyl ethylhexanoate, C₁₂₋₁₃ alkyl lactate, C₁₂₋₁₅ alkyl lactate, C₂₀₋₂₄ alkyl methicone, C₂₄₋₂₈ alkyl methicone, calodendrum capense nut oil, calophyllum tacamahaca seed oil, cetearyl dimethicone/vinyl dimethicone crosspolymer, cetearyl ethylhexanoate, cetearyl isononanoate, cetearyl nonanoate, cetearyl palmitate, cetrimonium laureth-12 succinate, cetyl acetate, cetyl caprylate, cetyl C₁₂₋₁₅ pareth-8 carboxylate, cetyl dimethicone, cetyl dimethicone/bis-vinyldimethicone crosspolymer, cetyl dimethyloctanoate, cetyl esters, cetyl ethylhexanoate, cetyl glyceryl ether, cetyl glycol, cetyl glycol isostearate, cetyl isononanoate, cetyl lactate, cetyl laurate, cetyl oleate, cetyloxy dimethicone, C₁₂₋₁₅ pareth-3 benzoate, C₁₂₋₁₅ pareth-9 hydrogenated tallowate, C₁₁₋₁₅ pareth-3 oleate, C₁₂₋₁₅ pareth-12 oleate, C₁₁₋₁₅ pareth-3 stearate, C₁₁₋₁₅ pareth-12 stearate, dibutyl adipate, dibutyldecyl IPDI, dibutyloctyl IPDI, dibutyloctyl malate, dibutyloctyl sebacate, dibutyl sebacate, Ddi-C₁₂₋₁₅ alkyl adipate, di-C₁₂₋₁₅ alkyl fumarate, di-C₁₂₋₁₃ alkyl malate, di-C₁₂₋₁₅ alkyl maleate, di-C₁₂₋₁₃ alkyl tartrate, —C₁₄₋₁₅ alkyl tartrate, dicaprylyl carbonate, dicaprylyl ether, dicaprylyl maleate, dicetyl adipate, dicocoyl pentaerythrityl distearyl citrate, diethyl adipate, isobutyl myristate, isobutyl palmitate, isobutyl pelargonate, isobutyl stearate, isobutyl tallowate, isocetyl alcohol, isocetyl ethylhexanoate, isocetyl isodecanoate, isocetyl isostearate, isocetyl laurate, isocetyl linoleoyl stearate, isocetyl palmitate, isocetyl stearate, lanolin, lanolin oil, lanolin wax, lauryl lactate, neopentyl glycol diheptanoate, neopentyl glycol diisononanoate, neopentyl glycol dilaurate, octyldodecyl ethylhexanoate, octyldodecyl lactate, octyldodecyl neodecanoate, octyldodecyl neopentanoate, PPG-3 benzyl ether myristate, sunflower oil, safflower oil, mineral oil and jojoba oil diisoamyl malate, diethylhexyl malate, dibutyloctyl malate, dimethyl capramide, and combinations thereof.

As will be recognized by one skilled in the art, the relative amounts of such carriers in the skin protectant formulation of the disclosure will be dictated by the nature of the skin protectant formulation. The levels of carrier can be determined by routine experimentation in view of the disclosure provided herein, and may be present in the skin protectant formulation in amounts of from about 0.01 wt. % to about 70 wt. % and more desirably from about 0.1 wt. % to about 65 wt. %.

In exemplary implementations, the skin protectant formulation can also contain additional ingredients, or treatment agents, for treating the skin, hair or body. Typically, the formulation includes a treatment agent that is known to have a treating effect such as reducing fine lines and wrinkles, improving the evenness of skin tone, and reduction of acne. More specifically, the treatment agent can be selected from the group consisting of appearance modifying agents (e.g., tooth whitening agents, exfoliating agents, skin-firming agents, anti-callous agents, anti-acne agents, anti-aging agents, anti-wrinkle agents, anti-dandruff agents, antiperspirant agents, wound care agents, enzyme agents, scar repair agents, humectant agents, hair care agents such as conditioners, styling agents, and detangling agents), therapeutic agents, pharmaceuticals (e.g., drugs, transdermal drug delivery agents, magnets, magnetic metals, and foods), xenobiotics, skin coloration agents (e.g., tanning agents, lightening agents, and brightening agents, shine control agents and drugs); shine control agents, colorant agents, surface conditioning agents (e.g., pH adjusting agents, moisturizers, skin conditioners, exfoliation agents, shaving lubricants, skin-firming agents, anti-callous agents, anti-acne agents, anti-aging agents, anti-wrinkle agents, anti-dandruff agents, wound care agents, skin lipids, enzymes, scar care agents, humectants, powders, botanical extracts, and drugs) external analgesic agents, anti-inflammatory (e.g., anti-irritant agents, anti-allergy agents, wound care agents, transdermal drug delivery, and drugs), fragrances, odor neutralizing agents, soothing agents, calming agents, antiperspirants, deodorants, botanical extracts (e.g., peppermint oil, eucalyptol, eucalyptus oil, camphor, and tea tree oil), peptides, natural and synthetic fats or oils, moisture absorbers, and combinations thereof. Further examples of suitable ingredients include those described in CTFA, International Cosmetic Ingredient Dictionary and Handbook, 12th Ed. (2008).

The term “natural fat or oil” is intended to include fats, oils, essential oils, essential fatty acids, non-essential fatty acids, phospholipids, and combinations thereof. Suitable fats and oils include Apricot Kernel Oil, Avocado Oil, Babassu Oil, Borage Seed Oil, Butter, C₁₂₋₁₈ Acid Triglyceride, Camellia Oil, Canola Oil, Caprylic/Capric/Lauric Triglyceride, Caprylic/Capric/Linoleic Triglyceride, Caprylic/Capric/Stearic Triglyceride, Caprylic/Capric Triglyceride, Carrot Oil, Cashew Nut Oil, Castor Oil, Cherry Pit Oil, Chia Oil, Cocoa Butter, Coconut Oil, Cod Liver Oil, Corn Germ Oil, Corn Oil, Cottonseed Oil, C₁₀₋₁₈ Triglycerides, Egg Oil, Epoxidized Soybean Oil, Evening Primrose Oil, Glyceryl Triacetyl Hydroxystearate, Glyceryl Triacetyl Ricinoleate, Glycosphingolipids, Grape Seed Oil, Hazelnut Oil, Human Placental Lipids, Hybrid Safflower Oil, Hybrid Sunflower Seed Oil, Hydrogenated Castor Oil, Hydrogenated Castor Oil Laurate, Hydrogenated Coconut Oil, Hydrogenated Cottonseed Oil, Hydrogenated C₁₂₋₁₈ Triglycerides, Hydrogenated Fish Oil, Hydrogenated Lard, Hydrogenated Menhaden Oil, Hydrogenated Mink Oil, Hydrogenated Orange Roughy Oil, Hydrogenated Palm Kernel Oil, Hydrogenated Palm Oil, Hydrogenated Peanut Oil, Hydrogenated Shark Liver Oil, Hydrogenated Soybean Oil, Hydrogenated Tallow, Hydrogenated Vegetable Oil, Lanolin and Lanolin Derivatives, Lard, Lauric/Palmitic/Oleic Triglyceride, Lesquerella Oil, Linseed Oil, Macadamia Nut Oil, Maleated Soybean Oil, Meadowfoam Seed Oil, Menhaden Oil, Mink Oil, Moringa Oil, Mortierella Oil, Neatsfoot Oil, Oleic/Linoleic Triglyceride, Oleic/Palmitic/Lauric/Myristic/Linoleic Triglyceride, Oleostearine, Olive Husk Oil, Olive Oil, Omental Lipids, Orange Roughy Oil, Palm Kernel Oil, Palm Oil, Peach Kernel. Oil, Peanut Oil, Pengawar Djambi Oil, Pentadesma Butter, Phospholipids, Pistachio Nut Oil, Placental Lipids, Rapeseed Oil, Rice Bran Oil, Safflower Oil, Sesame Oil, Shark Liver Oil, Shea Butter, Soybean Oil, Sphingolipids, Sunflower Seed Oil, Sweet Almond Oil, Tall Oil, Tallow, Tribehenin, Tricaprin, Tricaprylin, Triheptanoin, Trihydroxymethoxystearin, Trihydroxystearin, Triisononanoin, Triisostearin, Trilaurin, Trilinolein, Trilinolenin, Trimyristin, Trioctanoin, Triolein, Tripalmitin, Trisebacin, Tristearin, Triundecanoin, Vegetable Oil, Walnut Oil, Wheat Bran Lipids, Wheat Germ Oil, Zadoary Oil, oil extracts of various other botanicals, and other vegetable or partially hydrogenated vegetable oils, and the like, as well as mixtures thereof. In addition to being a skin treatment agent, the natural fats or oils may also be hydrophobic carriers.

Suitable fatty acids include Arachidic Acid, Arachidonic Acid, Behenic Acid, Capric Acid, Caproic Acid, Caprylic Acid, Coconut Acid, Corn Acid, Cottonseed Acid, Hydrogenated Coconut Acid, Hydrogenated Menhaden Acid, Hydrogenated Tallow Acid, Hydroxystearic Acid, Isostearic Acid, Lauric Acid, Linoleic Acid, Linolenic Acid, Linseed Acid, Myristic Acid, Oleic Acid, Palmitic Acid, Palm Kernel Acid, Pelargonic Acid, Ricinoleic Acid, Soy Acid, Stearic Acid, Tall Oil Acid, Tallow Acid, Undecanoic Acid, Undecylenic Acid, Wheat Germ Acid, and the like, as well as mixtures thereof.

Suitable essential oils include Anise Oil, Balm Mint Oil, Basil Oil, Bee Balm Oil, Bergamot Oil, Birch Oil, Bitter Almond Oil, Bitter Orange Oil, Calendula Oil, California Nutmeg Oil, Caraway Oil, Cardamom Oil, Chamomile Oil, Cinnamon Oil, Clary Oil, Cloveleaf Oil, Clove Oil, Coriander Oil, Cypress Oil, Eucalyptus Oil, Fennel Oil, Gardenia Oil, Geranium Oil, Ginger Oil, Grapefruit Oil, Hops Oil, Hyptis Oil, Indigo Bush Oil, Jasmine Oil, Juniper Oil, Kiwi Oil, Laurel Oil, Lavender Oil, Lemongrass Oil, Lemon Oil, Linden Oil, Lovage Oil, Mandarin Orange Oil, Matricaria Oil, Musk Rose Oil, Nutmeg Oil, Olibanum, Orange Flower Oil, Orange Oil, Patchouli Oil, Pennyroyal Oil, Peppermint Oil, Pine Oil, Pine Tar Oil, Rose Hips Oil, Rosemary Oil, Rose Oil, Rue Oil, Sage Oil, Sambucus Oil, Sandalwood Oil, Sassafras Oil, Silver Fir Oil, Spearmint Oil, Sweet Marjoram Oil, Sweet Violet Oil, Tar Oil, Tea Tree Oil, Thyme Oil, Wild Mint Oil, Yarrow Oil, Ylang Ylang Oil, and the like, as well as mixtures thereof.

The term “synthetic fat or oil” is intended to include synthetic fats and oils, esters, silicones, other emollients, and combinations thereof. Examples of suitable synthetic fats or oils include mineral oils, mineral jelly, isoparaffins, polydimethylsiloxanes such as methicone, cyclomethicone, dimethicone, dimethiconol, trimethicone, alkyl dimethicones, alkyl methicones, alkyldimethicone copolyols, organo-siloxanes (i.e., where the organic functionality can be selected from alkyl, phenyl, amine, polyethylene glycol, amine-glycol, alkylaryl, carboxal, and the like), silicones such as silicone elastomer, phenyl silicones, alkyl trimethylsilanes, dimethicone crosspolymers, cyclomethicone, gums, resins, fatty acid esters (esters of C₆₋₂₈ fatty acids and C₆₋₂₈ fatty alcohols), glyceryl esters and derivatives, fatty acid ester ethoxylates, alkyl ethoxylates, C₁₂₋₂₈ fatty alcohols, C₁₂₋₂₈ fatty acids, C₁₂₋₂₈ fatty alcohol ethers, propylene glycol esters and derivatives, alkoxylated carboxylic acids, alkoxylated alcohols, Guerbet alcohols, Guerbet Acids, Guerbet Esters, and other cosmetically acceptable emollients. In addition to being a skin treatment agent, the synthetic fat or oils may also be hydrophobic carriers.

Specific examples of suitable esters may include, but are not limited to, cetyl palmitate, stearyl palmitate, cetyl stearate, isopropyl laurate, isopropyl myristate, isopropyl palmitate, and combinations thereof.

In another aspect, the skin protectant typically moisturizes the skin and/or provides a barrier to minimize loss of moisture from the skin. Specifically, the treatment agent can be any moisturizing agent and/or moisture-barrier enhancing agent known in the art.

The content of the treatment agent in the liquid skin protective formulation is preferably not less than 0.01 to about 40 wt.%, and more preferably from 0.05 to about 30 wt.%.

The skin protectant formulation of the present disclosure could include barrier, perineal, moisturizing, cleansing, shampoo, liquid powder, anti-itch formulations used to treat human skin. In one exemplary aspect, the skin protectant formulation may be used for treatment of diaper or incontinence dermatitis caused by one or more of occlusion, chafing, moisture, mechanical or chemical irritation, or continued exposure to urine or feces.

The following non-limiting example is provided to further illustrate the present disclosure.

In this example, a skin protectant formulation was prepared. The following ingredients were used to prepare the skin protectant formulation.

| Trade Name | INCI Name | Wt. % | Grams |
|---|---|---|---|
| Phase 1 | | | |
| Water | Water | 67.18 | 335.90 |
| Laponite XLG | Magnesium Aluminum Silicate | 1.70 | 8.50 |
| Citric Acid | Citric Acid | 0.62 | 3.10 |
| Sonnecone CM | Petrolatum | 30.00 | 150.00 |
| Phenonip XB | Phenoxyethanol, Methylparaben, | 0.50 | 2.50 |
| | Propylparaben, Ethylparaben | | |

To prepare the exemplary formulation, the Laponite was added to water and stirred at high speed for 20 minutes or until hydrated. Then, citric acid was added and stirred at high speeds for 15 minutes. The resultant mixture was then placed into a homogenizer. Petrolatum was then added under high shear mixing, and the formulation was allowed to mix until homogeneous. Finally, preservative was added and mixing was continued for 5 minutes.

As shown above, the skin protectant formulation contains laponite clay as a structurant and Sonnecone CM petrolatum as the skin protectant. Phenonip XB is a preservative blend containing phenoxyethanol, ethylparaben, methylparaben and propylparaben.

The formulation was then added to a refillable continuous spray-type dispenser. The container was pumped approximately 20 times to place the formulation under pressure. The spray button was then pushed and the formulation sprayed in a fine mist through the dispenser.

When introducing elements of the present disclosure, the articles “a”, “an”, “the” and “said” are intended to mean that there are one or more of the elements. The terms “comprising”, “including” and “having” are intended to be inclusive and mean that there may be additional elements other than the listed elements.

In view of the above, it will be seen that the several aspects of the disclosure are achieved and other advantageous results attained.

As various changes could be made in the above formulations and products without departing from the scope of the disclosure, it is intended that all matter contained in the above description shall be interpreted as illustrative and not in a limiting sense.

1. A sprayable skin protection formulation comprising:
a carrier;
a structurant; and
at least about 30 wt. % of a skin protectant agent.

2. The sprayable skin protection formulation of claim 1 wherein the skin protectant agent is selected from mineral oil, dimethicone, zinc oxide, allantoin, calamine, kaolin, petrolatum, white petrolatum, cod liver oil, lanolin, talc, topical starch, aluminum hydroxide gel, cocoa butter, glycerine, shark liver oil, zinc acetate, zinc carbonate, and combinations thereof.

3. The sprayable skin protection formulation of claim 1 wherein the skin protectant agent is petrolatum.

4. The sprayable skin protection formulation of claim 1 comprising between about 30 wt. % to about 60 wt. % of a skin protectant agent.

5. The sprayable skin protection formulation of claim 1 wherein the skin protectant agent is lanolin, mineral oil or cocoa butter.

6. The sprayable skin protection formulation of claim 1 wherein structurant provides a network trapping the skin protectant agent within the network.

7. The sprayable skin protection formulation of claim 1 wherein the structurant is selected from an acrylic polymer, a clay, a starch, a modified cellulose, a natural gum, a wax, colloidal particles, and/or mixtures thereof.

8. The sprayable skin protection formulation of claim 6 wherein the structurant comprises the clay, the clay comprising bentonite, laponite, hectorite, montmorillonite, beidelite, saponite, stevensite, magnesium aluminum silicate, other aluminum silicates, as well as various other natural and/or synthetic clays, and/or combinations thereof.

9. The sprayable skin protection formulation of claim 6 wherein the structurant is laponite clay.

10. A sprayable skin protection formulation of claim 6, wherein the structurant comprises a starch.

11. A sprayable skin protection formulation of claim 6, wherein the modified cellulose comprises ethyl cellulose, hydroxypropyl cellulose, carboxymethyl cellulose, hydroxypropyl methyl cellulose, hydroxyethyl methyl cellulose, hydroxyethyl cellulose, and/or mixtures thereof.

12. A sprayable skin protection formulation of claim 6 wherein the natural gum comprises guar gum, pectin, gum Arabic, locust bean gum, xanthan gum, carrageenan gum, and/or mixtures thereof.

13. The sprayable skin protection formulation of claim 1 comprising from about 0.01 wt. % to about 10 wt. % of the structurant.

14. The sprayable skin protection formulation of claim 1 comprising from about 0.05 wt. % to about 5 wt. % of the structurant.

15. The sprayable skin protection formulation of claim 1 comprising from about 0.1 wt. % to less than about 5 wt. % of the structurant.

16. The sprayable skin protection formulation of claim 1 wherein the carrier is selected from a hydrophilic carrier, a hydrophobic carrier or an emulsion.

17. The sprayable skin protection formulation of claim 15 wherein the hydrophilic carrier is water.

18. The sprayable skin protection formulation of claim 1 further comprising a treatment agent.

19. The sprayable skin protection formulation of claim 18 wherein the treatment agent is between about 0.01 to about 40 wt. % of the skin protectant formulation.

20. The sprayable skin protection formulation of claim 18 wherein the treatment agent is between about 0.05 to about 30 wt. % of the skin protectant formulation.

21. The sprayable skin protection formulation of claim 18 further comprising a treatment agent selected from the group consisting of emollients, humectants, natural fats and oils, anti-irritants, antimicrobial agents, antioxidants, anti-parasitic agents, antipuritics, antifungals, antiseptic actives, keratolytic actives, anti-stinging agents, anti-reddening agents, astringents, biological actives, deodorants, external analgesics, film formers, fragrances, skin condition agents, skin exfoliating agents, skin protectants, skin soothing ingredients, sunscreens and combinations thereof.

22. A sprayable skin protection formulation comprising:
a hydrophilic carrier, the hydrophilic carrier being water;
a structurant, wherein the structurant is a laponite clay; and
at least about 30 wt. % of a skin protectant agent, wherein the skin protectant agent is petrolatum.

23. The sprayable skin protection formulation of claim 22 comprising between about 30 wt. % to about 60 wt. % of a skin protectant agent.

24. A method of treating fragile and sensitive skin comprising:
providing a sprayable spray skin protection formulation comprising a carrier, a structurant; and at least about 30 wt. % of a skin protectant agent; and
continuously spraying the skin protectant formulation onto an area of human skin for protecting the skin.

25. The method of claim 24 wherein the skin protectant agent is petrolatum.

26. The method of claim 24 further comprising applying a fine mist of the skin protection formulation onto the human skin.

27. The method of claim 24 further comprising applying droplets of the skin protection formulation onto the skin for application without the need for excessive rubbing.
